(19) United States
(12) Patent Application Publication
(10) Pub. No.: US 2017/0042636 A1
(43) Pub. Date: Feb. 16, 2017

(54) DISINFECTING DEVICE

(71) Applicant: THE QUEEN ELIZABETH HOSPITAL KING'S LYNN NHS FOUNDATION TRUST, Kings Lynn (GB)

(72) Inventor: Peter Young, Roydon, Kings Lynn (GB)

(21) Appl. No.: 15/118,599

(22) PCT Filed: Feb. 14, 2014
(86) PCT No.: PCT/GB2014/050447

§ 371 Date: Aug. 12, 2016

(51) Int. Cl. A61B 90/70; A61L 2/18; B08B 9/032; A61M 39/16; A61M 5/178

(52) U.S. Cl. CPC A61B 90/70; A61M 39/16; A61M 5/178; B08B 9/0321; A61L 2/18; A61B 2090/701; A61L 2202/24

(57) ABSTRACT

A device (1) for disinfecting a port of a medical device in fluid communication with a patient is disclosed. The device comprises a portion (2) adapted for connection to the port. The external surface of the portion is coated with a composition (5) in a solid or solid-like state that comprises a disinfectant, and the disinfectant composition is able to release the disinfectant upon contact with a liquid. A method for disinfecting a port of a medical device in fluid communication with a patient is also disclosed.

### FIELD OF THE INVENTION

The present invention relates to a disinfecting device. In particular, the present invention relates to a device and a method for disinfecting a medical device.

### BACKGROUND

Vascular access devices (VADs), such as cannulae and catheters, are used to supply patients with pharmaceuticals, liquid nourishment, or blood products.

Connector hubs are commonly used to facilitate docking of devices used to administer and/or withdraw fluid from a VAD. Connector hubs often have a valve or tap-like system. Examples include needle-free connectors and three-way taps.

However, such connector hubs are susceptible to contamination by bacteria and other microorganisms when they are not in use. In a number of studies, needle-free connector hubs have been shown to be internally colonised with bacteria, and some studies have shown that hub colonisation is a major source of catheter-related bloodstream infections (Ramritu et al., 2008; Richardson et al., 2013; Maragakis et al., 2006; Jarvis et al., 2009; Yebenes et al., 2004; Esteve et al., 2007; and Yebenes et al., 2008).

Bacterial contamination of the connector hubs may be the result of a poor adherence to the disinfection protocol prior to use. However, bacterial colonisation of the connector hub can still take place even when the recommended disinfection procedures are followed.

Furthermore, it has been demonstrated in the literature that the microbial colonisation of catheter hubs may be due to the common use of contaminated syringes (Austin and Elia, 2013; Kerenyi et al., 2011; van Grafhorst et al., 2002; Stucki et al., 2009 and Magee et al., 1995).

Once a connector hub has become contaminated, there is a high probability that the contaminating microbes will be transmitted to the patient. There are several reasons for this. Firstly, the same connector hub is normally in use for 72 hours, and often multiple injections are given via the connector hub during this time. In addition, clinical observation alone is not sufficient to detect contamination, so a contaminated connector hub may continue to be used. Lastly, once a connector hub has been contaminated, full disinfection of the connector hub using the standard disinfection procedure of wiping the connector hub with the recommended disinfectant can be difficult to achieve.

Solutions to this problem have been proposed, but these often involve complex assemblies which can be expensive to manufacture and/or difficult to use. The use of caps on the ports or the connector hubs has also been proposed, but this would still leave the port or connector hub susceptible to contamination when the cap is not in place. In addition, some ports and connector hubs are shaped so that they do not accept a cap.

The present invention aims to solve one or more of the above-mentioned problems.

### SUMMARY OF THE INVENTION

According to a first aspect, there is provided a device for disinfecting a port of a medical device in fluid communication with a patient, the device comprising a portion adapted for connection to the port, wherein:
the external surface of the portion is coated with a composition in a solid or solid-like phase that comprises a disinfectant, and wherein the disinfectant composition is able to release the disinfectant upon contact with a liquid.

In a further aspect, there is provided a syringe for disinfecting a port of a medical device in fluid communication with a patient, the syringe comprising a barrel and a tip, the syringe tip being adapted for connection to the port, wherein:
the external surface of the syringe tip is coated with a composition in a solid or solid-like phase that comprises a disinfectant, and wherein the disinfectant composition is able to release the disinfectant upon contact with a liquid.

The port may comprise a connector hub. In some embodiments, the medical device comprises a catheter and/or a cannula. In some embodiments, the medical device comprises a connecting tube.

The disinfectant composition may exclusively coat the external surface of the syringe tip. The entire surface area of the external surface of the syringe tip may be coated with the disinfectant composition. Alternatively, a fraction of the surface area of the external surface of the syringe tip may be coated with the disinfectant composition, for example one half of the external surface of the syringe tip may be coated with the disinfectant composition. In some embodiments, the barrel of the syringe is coated with the disinfectant composition.

In some embodiments, the syringe tip is adapted for the formation of a luer connection with the port. The luer connection may be a luer lock connection or a luer slip connection. In some embodiments, the syringe tip is adapted for the formation of a male luer connection and the port is adapted for the formation of a female luer connection.

In some embodiments, the disinfectant composition is formulated such that the disinfectant composition dissolves and/or disperses. Thus, the disinfectant composition is able to release the disinfectant upon contact with a liquid by the disinfectant dissolving or dispersing in the liquid.

The disinfectant may comprise one or more of a biocidal agent, a sporicidal agent, a bactericidal agent or a viricidal agent.

According to a second aspect, there is provided a process for disinfecting a port of a medical device in fluid communication with a patient comprising:
(a) placing the device or syringe according to the first aspect into the port; and
(b) contacting the disinfectant composition with a liquid, thereby releasing the disinfectant. Steps (a) and (b) may take place simultaneously.

According to a third aspect, there is provided a kit comprising the device or syringe according to the first aspect and a connector hub for use with a medical device to be placed in fluid communication with a patient.

### DRAWINGS

Embodiments of the disclosure will now be described, by way of example only, with reference to the accompanying drawings, in which:

FIG. 1 shows a perspective view of a needle-free syringe;

FIG. 2 shows a perspective view of the tip of a needle-free syringe, the tip having a disinfectant coating;

FIG. 3 shows a perspective view of the tip of a needle-free syringe, the tip having a disinfectant coating;

FIG. 4 shows a transverse cross-sectional view of the syringe tip of FIG. 2;

FIG. 5 shows a transverse cross-sectional view of the syringe tip of FIGS. 2 and 4 placed in contact with a port;

FIG. 6 is a graph showing the reduction in bacterial growth in needle-free connector hubs using the disinfecting device of FIGS. 2 and 4; and

FIG. 7 is a graph showing the reduction in bacterial growth in three-way taps using the disinfecting device of FIGS. 2 and 4.

### DETAILED DESCRIPTION

A device for disinfecting a port of a medical device that is in fluid communication with a patient is provided.

In some embodiments, the medical device comprises a vascular access device, e.g. a catheter or a cannula, which may be inserted into the arterial or venous system. More particularly, the medical device may comprise an intravenous or arterial catheter or cannula. In some embodiments, the medical device comprises a connecting tube.

The medical device or the port of the medical device may comprise a connector hub. As used herein, the term “connector hub” refers to any device that permits fluid flow between a source and the medical device. Connector hubs are routinely used to selectively facilitate the movement of fluid between two points. The term “connector hub” may be used interchangeably with the terms “hub” and/or “connector”. In some embodiments, the connector hub is an injector hub. In other embodiments, the connector hub is a needle-free connector. In yet other embodiments, the connector hub is a three-way tap. Thus, in embodiments where the medical device comprises a connector hub, the disinfecting device of the present invention disinfects a port of a connector hub. The term “port of the medical device” may be used interchangeably with the term “port of the connector hub”.

The disinfecting device of the present invention comprises a portion adapted for connection to the port of the medical device. More particularly, the portion is adapted such that, when the portion is connected to the port, the external surface of the portion is in contact with the internal surface of the port.

In some embodiments, the portion of the disinfecting device is adapted so that it forms a luer connection when it is placed in contact with the port. The luer connection may be a luer slip connection. Alternatively, the luer connection may be a luer lock connection. In some embodiments, the portion of the disinfecting device comprises a male luer connection and the port of the medical device comprises a female luer connection.

The disinfecting device may be any device that comprises a portion that is adapted for connection to the port of the medical device or connector hub. In some embodiments, the disinfecting device is for the administration and/or withdrawal of fluid from the medical device. For example, the disinfecting device may be used to administer fluids comprising medication and/or nutrients to the patient and/or to take samples of body fluids, such as blood samples, from a patient. Alternatively or in addition, the disinfecting device may be used to apply a flushing or wash solution to the medical device. In some embodiments, the disinfecting device is used for bladder washouts. In some embodiments, the disinfecting device is used to administer medication via a nasogastric tube. In some embodiments, the disinfecting device is used to administer medication such as an anaesthetic via an epidural or intrathecal route.

In some embodiments, the disinfecting device may be a tube or other medical device such as a conduit, syringe, IV set (both peripheral and central lines), piggyback line, or similar component which comprises a portion that is adapted for connection to the port of the medical device.

The disinfecting device may be made of any suitable material.

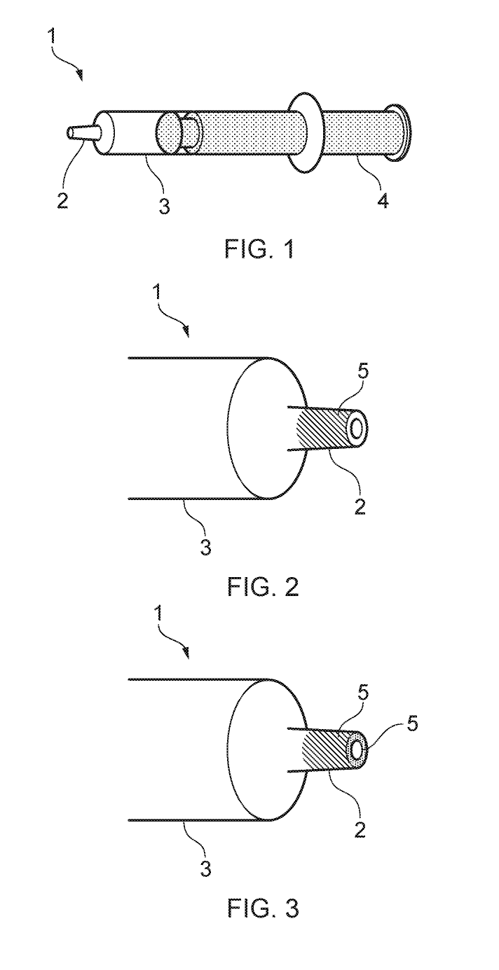

In some embodiments, the disinfecting device is a syringe. FIG. 1 shows a needle-free syringe 1 with a tip 2, barrel 3 and plunger 4. The syringe 1 may be any syringe that comes into contact with a port of a medical device. The syringe tip 2 can be considered to be equivalent to the portion of a disinfecting device adapted for connection to the port of the medical device.

Syringes are commonly drawn up with medicine in preparation and kept in non-sterile containers until used to inject into the patient. A syringe having a disinfectant coating offers the advantage that the syringe may be placed in a non-sterile container with a minimum risk of the syringe being contaminated before use. This also prevents contamination of the syringe during clinical procedures in which the syringe is left in a non-sterile environment for considerable periods of time. For example, during the performance of general anaesthesia or resuscitative care, incremental doses of a drug from a single syringe may be titrated to effect over many hours.

The syringe tip 2 in FIG. 1 is tapered. However, the external surface of the portion of the disinfecting device may have any shape provided that it is adapted for connection to the port.

The external surface of the portion of the device is coated with a composition in a solid or solid-like phase that comprises a disinfectant. In some embodiments, the external surface of the portion of the device is coated with a composition in a solid phase. As used herein, the term “disinfectant coating” may be used interchangeably with the term “disinfectant composition”, and refers to a composition in a solid or solid-like phase, preferably solid phase, that comprises one or more disinfectant(s). The disinfectant coating is located on the external surface of the portion of the disinfecting device that is adapted for connection to the port.

In some embodiments, the external surface of the tip of a syringe is coated with the composition. This is illustrated in FIG. 2, in which the barrel 3 and tip 2 of syringe 1 are shown, and the disinfectant coating 5 is illustrated by the shading on the syringe tip 2.

In FIG. 2, the disinfectant coating 5 covers the majority of the external cylindrical wall of the syringe tip 2. In some embodiments, the disinfectant coating 5 coats all or substantially all of the external cylindrical wall of the syringe tip 2. In some embodiments, the disinfectant coating 5 coats a reduced area of the external cylindrical wall of the syringe tip 2 than shown in FIG. 2.

FIG. 3 shows an alternative embodiment, in which the syringe tip 2 is coated with the disinfectant coating 5 on the cylindrical wall and on the front-end of the syringe tip 2. In some embodiments, the disinfectant coating 5 coats all or substantially all of the external cylindrical wall of the syringe tip 2. In some embodiments, the disinfectant coating 5 coats a reduced area of the external cylindrical wall of the syringe tip 2 than shown in FIG. 3.

In another alternative embodiment, the front-end of the syringe tip 2 may be coated but not the cylindrical wall of the syringe tip 2.

In some embodiments, the disinfectant coating is applied to a more extensive area than the external surface of the portion that is adapted to be placed in contact with the port. For example, in the embodiment shown in FIG. 2, in addition to the syringe tip 2 the disinfectant coating may also be applied to the barrel 3 of the syringe 1, which may offer the advantage of ease of manufacture. Alternatively or in addition, having the disinfectant coating on a more extensive area than the external surface of the portion that is adapted to be placed in contact with the port may offer the advantage of improving hand disinfection in the clinical environment.

In some embodiments, the disinfectant coating is on the external surface of the portion that is adapted to be placed in contact with the port, and no other part of the disinfecting device. Thus, in some embodiments in which the disinfecting device is a syringe 1, the disinfectant coating exclusively coats the syringe tip 2.

In some embodiments, the disinfectant coating is applied to the entire surface area of external surface of the portion of the disinfecting device. In some embodiments, the disinfectant coating is applied to a proportion or a fraction of the surface area of the external surface of the portion of the disinfecting device. For example, the disinfectant may be applied to about one quarter, one third, one half, two thirds or three quarters of the surface area of the external surface of the portion of the disinfecting device.

The disinfectant coating may cover an area of the external surface of the portion of the disinfecting device of a sufficient size to carry a sufficient amount of disinfectant to disinfect the port.

The disinfectant coating may be a continuous layer on the external surface. Alternatively, the disinfectant coating may be a non-continuous layer. For example, the disinfectant coating may be in the form of a pattern such as one or more ring(s) and/or one or more stripe(s).

The external surface of the portion of the disinfecting device that is coated with the disinfectant composition may be distinguished from the non-coated surface. For example, the coated surface may be a different colour from the non-coated surface and/or may have a different surface texture from the non-coated surface. This may be particularly advantageous for a situation in which both disinfecting and non-disinfecting devices are used, for example disinfecting and non-disinfecting syringes, to assist the user in distinguishing between the two devices.

As used herein, the term “disinfectant” refers to an agent that has antimicrobial activity and is applied to non-living objects.

The disinfectant may have immediate antimicrobial activity. As used herein, the term “immediate antimicrobial activity” refers to the disinfectant having an effect on microorganisms immediately, for example within one minute.

In some embodiments, the disinfectant has persistent antimicrobial activity. As used herein, the term “persistent antimicrobial activity” refers to the disinfectant having a prolonged antimicrobial effect after the disinfecting device has first been applied to the port.

The disinfectant may have antimicrobial activity for at least 1 hour, at least 2 hours, at least 3 hours, at least 4 hours, at least 5 hours, at least 6 hours, at least 7 hours, at least 8 hours, at least 9 hours and/or at least 10 hours after the disinfecting device has first been applied to the port.

The disinfectant may have antimicrobial activity for up to 1 hour, up to 2 hours, up to 3 hours, up to 4 hours, up to 5 hours, up to 6 hours, up to 7 hours, up to 8 hours, up to 9 hours and/or up to 10 hours after the disinfecting device has first been applied to the port.

In some embodiments, the disinfectant has residual antimicrobial activity. As used herein, the term “residual antimicrobial activity” refers to the disinfectant having an antimicrobial effect when the disinfecting device is used repeatedly.

The disinfectant comprises one or more antimicrobial agent(s) which destroys or kills microorganisms. Examples of antimicrobial agents include antibacterial agents, antiviral agents and/or antifungal agents. The antimicrobial agent may comprise a biocidal agent, a sporicidal agent, a bactericidal agent and/or a viricidal agent.

The disinfectant may additionally inhibit the growth of microorganisms. In these embodiments, the antimicrobial agent may comprise a biostatic agent, a bacteriostatic agent and/or a sporistatic agent.

In some embodiments, the disinfectant comprises an antibiotic. In some embodiments, the disinfectant does not comprise an antibiotic. In some embodiments, the disinfectant does not comprise a therapeutic agent and/or an agent that has a therapeutic effect.

As used herein, the term “therapeutic agent” is used to refer to an agent that is used for the medical treatment of a patient, for example for the treatment of a disease or a medical condition. The therapeutic agent may have a curative and/or a preventative effect. The therapeutic agent may have been the subject of medical authorisation for its use in the treatment of a patient.

The disinfectant coating may comprise a disinfectant that has been approved for clinical use. The use of such disinfectants in the present invention offers the advantage that the properties of the disinfectant that are relevant in a clinical setting, such as the efficacy of the disinfectant and its potential allergenic effect, are known.

In some embodiments, the disinfectant coating may comprise a disinfectant that does not have an adverse effect on the patient if it comes into contact with the fluid flow from the medical device and the patient.

In some embodiments, the disinfectant comprises chlorhexidine. In some embodiments, the disinfectant consists essentially of chlorhexidine. In some embodiments, the disinfectant comprises silver or silver compounds.

In some embodiments, the disinfectant comprises chlorhexidine, silver, silver nitrate, silver sulfadiazine, glutaraldehyde, o-Phthalaldehyde, alexidine, polyhexamethylene biguanides (PHMB), sodium hypochlorite, chlorine dioxide, N-chloro compounds, hydrogen peroxide, peracetic acid, and/or one or more alcohol(s). Examples of suitable alcohols include ethanol and isopropyl alcohol. In some embodiments, the disinfectant comprises one or more substance(s) or compound(s) that can be converted into an alcohol. For example, the one or more substance(s) or compound(s) may be converted into an alcohol on contact with a liquid.

In some embodiments, the disinfectant comprises an antibiotic agent or a combination of two or more antibiotic agents. Examples of suitable antibiotic agents include gentamycin and neomycin.

The amount of disinfectant in the coating may be determined so that it is sufficient to disinfect the port and is present in acceptable levels for use in a clinical setting.

The disinfectant composition may be applied in one or more coatings or layers. In some embodiments, at least one coating or layer of the disinfectant composition is applied. In some embodiments, at least two coatings or layers of the disinfectant composition is applied.

In some embodiments, the disinfectant composition is applied as a liquid. In some embodiments the liquid disinfectant composition is in the form of a solution. The solution may comprise the disinfectant and a solvent. The solvent may be any solvent in which the disinfectant dissolves at the required temperature, at room temperature for example. In some embodiments, the solvent comprises an alcohol, such as ethanol and/or isopropyl alcohol. In some embodiments, the solvent comprises water. In some embodiments, the liquid disinfectant composition is in the form of a suspension.

In embodiments in which the disinfectant composition comprises or consists essentially of chlorhexidine, the disinfectant composition may be applied as a liquid. In some embodiments, the liquid comprises about 2% or about 4% chlorhexidine. In some embodiments in which the disinfectant composition comprises or consists essentially of chlorhexidine, isopropyl alcohol is used as a solvent.

In some embodiments, the disinfectant composition is applied as a solid. The disinfectant composition may be in the form of a powder, a granular material and/or fine particles. The particle or granule size may be substantially uniform. Thus, in embodiments in which the disinfectant composition comprises an antibiotic, the antibiotic may be in powder, granule or fine particle form. Similarly, in embodiments in which the disinfectant comprises silver, the silver may be in powder, granule or fine particle form.

In some embodiments, the disinfectant composition is applied as a gel. Thus, in embodiments in which the disinfectant composition comprises silver, the silver may be in a gel form, such as a hydrogel. Similarly, in embodiments in which the disinfectant composition comprises an alcohol, the alcohol may be in the form of a gel.

Suitable methods for applying the disinfectant composition to the external surface of the portion of the disinfecting device will be known to the person skilled in the art. The disinfectant composition may be applied by dipping and/or rolling the portion of the disinfecting device in the disinfectant composition. Alternatively or in addition, the disinfectant composition may be applied by spraying, sprinkling, brushing, wiping and/or printing the disinfectant composition onto the disinfecting device. For example, the disinfectant composition may be applied using pad printing. In some embodiments, the disinfectant composition is applied using electrostatic processes, such as electrostatic spraying or electrostatic powder coating. In some embodiments, the disinfectant composition is applied by deposition or electrodeposition.

In some embodiments, the external surface of the portion of the disinfecting device may be coated with a primer layer, and the disinfectant composition is applied to the primer layer. The primer layer may assist in the subsequent application of the disinfectant composition.

In some embodiments in which the disinfectant composition is applied as a solid, the external surface of the portion of the disinfecting device may be coated with an adhesive agent. The adhesive agent may be applied before and/or at the same time as the disinfectant composition. The adhesive agent may be sufficiently tacky or sticky for the disinfectant composition to adhere to the adhesive, whilst still allowing the disinfectant to be released during use. Suitable adhesive agents will be known to the person skilled in the art.

The disinfectant composition may be applied to form a coating or layer with a uniform or substantially uniform thickness. The coating or layer may have a thickness sufficient to carry a sufficient amount of disinfectant to disinfect the port.

In some embodiments, the disinfectant composition completely coats the external surface of the portion of the disinfecting device. In some embodiments, the disinfectant composition partially coats the external surface of the portion of the disinfecting device. Partial coating may be achieved by using masking techniques, for example.

In some embodiments, the external surface is coated with two or more, for example 3, 4, 5 or 6, different disinfectant compositions. The different disinfectant compositions may be spatially separated from each other on the external surface of the portion of the disinfecting device, For example, the different disinfectant compositions may be in the form of separate patches, stripes and/or rings on the external surface of the portion of the disinfecting device

Alternatively, the two or more, for example 3, 4, 5, or 6, different disinfectant compositions may not be spatially separated. For example, the disinfectant coating may comprise two or more disinfectant compositions in the form of a powder, a granular material and/or fine particles, the powders, granular materials and/or fine particles from the different disinfectant compositions being mixed together. In other words, the powders, granular materials and/or fine particles from the different disinfectant compositions are in close physical proximity.

In embodiments in which the disinfectant composition is applied as a liquid, the disinfectant composition dries to form a solid or a solid-like state. During this drying step, solvent present in the liquid disinfectant composition may be evaporated. The disinfectant composition may be dried by any known drying method. In some embodiments, the drying step comprises drying at room temperature. In some embodiments, the drying step comprises drying at an elevated temperature, for example in an oven.

In some embodiments, the external surface to which the disinfectant composition is applied has one or more grooves or depressions to facilitate application of the disinfectant composition to the surface.

Following the application of the disinfectant composition to the disinfecting device and the optional drying step, the disinfectant composition is in a solid phase or a solid-like phase, such as a gel. More particularly, the disinfectant composition is in a solid phase.

Devices comprising disinfectant in a liquid phase can require additional features such as sealed chambers to contain the disinfectant. Such chambers can increase the cost of production of the device and/or make the device cumbersome to use. In addition, devices containing liquid disinfectant are susceptible to the disinfectant evaporating from the device, which may render the device unusable. The disinfectant being in the liquid phase may therefore reduce the shelf-life of the device.

When a disinfectant is in a solid or solid-like phase it offers the advantage that the disinfectant is not susceptible to evaporation. The shelf life of the disinfecting device may therefore be increased in comparison to devices in which the disinfectant in the liquid phase. Furthermore, the disinfectant being in a solid or solid-like phase removes the requirement for the device to have additional features to prevent the disinfectant from evaporating, and thus the manufacture of the device can be cost effective.

The disinfectant composition may release the disinfectant. The disinfectant composition may release the disinfectant upon contact with a liquid. The liquid may comprise or consist essentially of water. Alternatively or in addition, the liquid may comprise an alcohol such as ethyl alcohol, optionally isopropyl alcohol, and/or any other alcohol or pre-alcohol liberating substance.

In embodiments in which the disinfectant coating comprises two or more different disinfectant compositions which are spatially separated on the external surface of the portion, the liquid may release the disinfectant compositions, thereby enabling the two compositions to combine. The disinfectant compositions may have a higher antimicrobial activity when combined than when separated.

In embodiments in which the disinfectant dissolves and/or disperses in the liquid, all or substantially all of the disinfectant coating may dissolve and/or disperse in the liquid. Alternatively, a proportion of the disinfectant coating, for example about one quarter, one third, one half, two thirds or three quarters of the disinfectant coating, may dissolve and/or disperse in the liquid. The retention of a proportion of the disinfectant coating on the external surface of the portion of the disinfecting device may offer the advantage that the disinfecting device may have residual and/or persistent antimicrobial activity after it has been applied to a port and/or may be used for repeat applications.

Once the disinfectant composition has been applied to the disinfecting device and is in a solid or solid-like phase, it may be in a pre-activated state. As used herein, the term “pre-activated state” is used to refer to the disinfectant having a lower antimicrobial activity than when the disinfectant is in the active state. In some embodiments, the disinfectant has little or no antimicrobial activity in the pre-activated state. Thus, the disinfectant composition may have little or no antimicrobial activity when in the solid or solid-like phase.

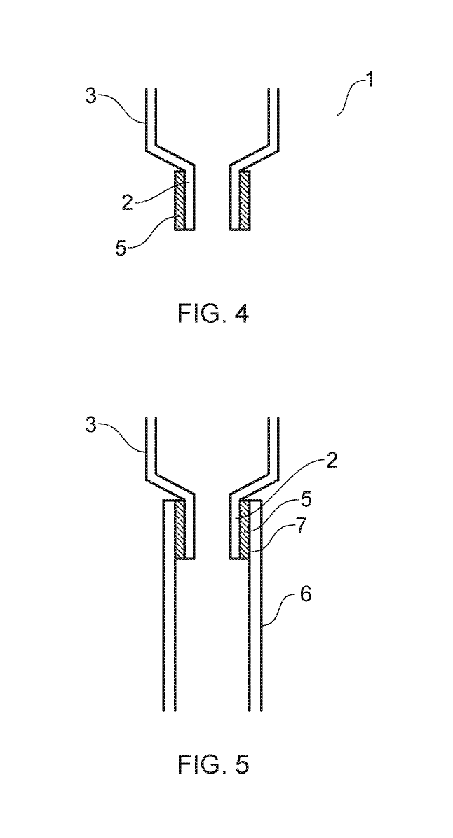

FIG. 4 shows a transverse cross-sectional view of syringe 1 with the external surface of the syringe tip 2 having a disinfectant coating 5. The syringe tip 2 has not been in contact with the port of a medical device, and thus in this embodiment the disinfectant is in a pre-activated state.

When the disinfecting device is in contact with the port of a medical device, the disinfectant may be in an active state. As used herein, the term “active state” is used to refer to the disinfectant having a higher antimicrobial activity than when the disinfectant is in the pre-activated state.

The disinfectant may be activated from a pre-activated state to an active state as a result of the disinfectant coating contacting a liquid. In these embodiments, the disinfectant coating may dissolve and/or disperse in the liquid, thereby releasing the disinfectant into the surrounding area. Thus, the disinfectant may be in a solution or suspension in the active state.

The disinfectant may be activated immediately prior to being placed in contact with the port of a medical device. For example, the disinfectant may be activated by the application of liquid to the disinfectant coating immediately prior to being placed in contact with the port of a medical device.

Alternatively or in addition, the disinfectant may be activated when it is placed in contact with the port of a medical device. For example, liquid within the port may activate the disinfectant, by dissolving and/or dispersing the disinfectant coating so as to release the disinfectant into the surrounding area. Thus, in this case, activation of the disinfectant takes places at the same time or at substantially the same time as the coated external surface of the portion of the disinfecting device being placed in contact with the port.

FIG. 5 shows a transverse cross-sectional view of syringe 1 with the external surface of the syringe tip 2 having a disinfectant coating 5. The syringe has been inserted into a port 6 of a medical device. The external surface of syringe tip 2 that has a disinfectant coating 5 is in contact with an internal surface of wall 7 of the port 6. The liquid within the port activates the disinfectant by dissolving and/or dispersing the disinfectant coating so as to release the disinfectant into the surrounding area. The disinfectant is therefore in an active state.

By having an external surface coated with an active disinfectant that comes into contact with the port, the desterilising device can decontaminate a port beyond the area that is decontaminated with standard practice. The use of the disinfecting device may even remove the requirement for additional disinfection protocols, such as standard wiping disinfection procedures.

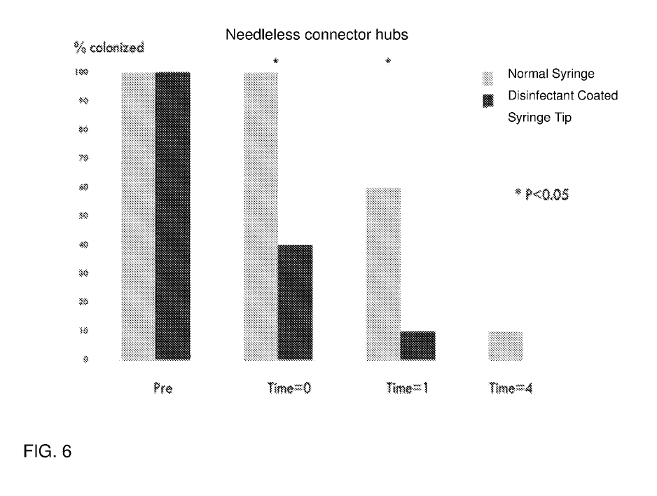

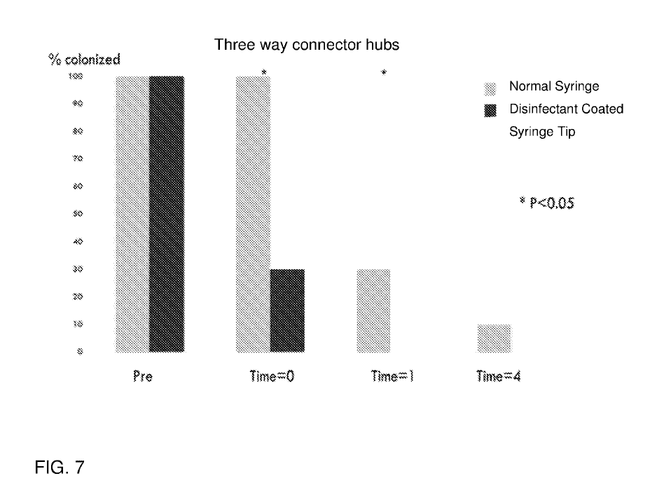

Having the disinfectant in the active state in the port has the effect that the antimicrobial activity of the disinfectant kills or destroys microbes in the port and therefore prevents port contamination. The disinfecting device prevents contamination of ports, and also decontaminates ports that have already been contaminated, thereby preventing the progression of microbes through the fluid pathway and reducing the potential for onward microbial transfer to the patient. This effect can be seen in Example 4 and FIGS. 6 and 7, which demonstrate that a syringe with its tip coated with disinfectant comprising chlorhexidine can effectively disinfect three-way and needle-less connectors. In comparison, the levels of contamination of the connectors were higher when non-coated control syringes were used.

In some embodiments, the disinfecting device may reduce the risk of catheter-related blood stream infection (CRBSI).

The disinfecting device effectively disinfects the port of medical devices, thereby reducing the risk of infection of the patient, without any change in clinical practice or increase in clinician workload. Moreover, as the disinfecting device is easy to use, it also has minimal implications for clinician training.

A process for disinfecting a port of a medical device in fluid communication with a patient is also provided by the present invention. The process may comprise decontaminating a contaminated port.

The process may comprise (a) placing the disinfecting device in the port of a medical device, so that the external surface of the portion of the disinfecting device comes into contact with the internal surface of the port, and (b) activating the disinfectant in the disinfectant coating to form a disinfectant in an active state. The disinfectant may be activated by contacting the disinfectant coating with a liquid. In these embodiments, the disinfectant coating may dissolve and/or disperse in the liquid. Steps (a) and (b) can be carried out in any order.

Activation of the disinfectant may comprise contacting the disinfectant with a liquid. In some embodiments, the activating liquid is within the port of the medical device, and therefore the disinfectant is activated when the disinfecting device is placed in contact with the port. In other words, activation of the disinfectant and placing the disinfecting device in contact with the port take place simultaneously.

One or more disinfecting devices may be provided in the form of a kit. The kit may further comprise a medical device to be placed in fluid communication with a patient, the medical device having a port. Alternatively or in addition, the kit may comprise one or more connector hub(s) to be placed on the port of a medical device in fluid communication with a patient.

In the context of the present disclosure other examples and variations of the devices and methods described herein will be apparent to a person of skill in the art.

### EXAMPLES

### Example 1

### Clinical Audit

The prevalence of bacterial colonisation of needle-free connectors and conventional three-way taps was studied.

A needle-free connector (SmartSite®, Carefusion) or a three-way tap (Becton Dickinson) that had been connected to an intravenous catheter in use by a patient was removed and cleaned in accordance with the manufacturer's instructions, by cleaning the connector surface with 2% chlorhexidine in 70% isopropyl wipes (Sani-Cloth®, PDI®) and allowing the surface to dry.

Following the drying period a microbiological swab was inserted into the connector hub to sample the area that would be in contact with a luer slip syringe tip. Swabbing was performed via a non-touch technique. Amies swabs (Sterilin®) were dipped in sterile purified water and inserted into the internal part of the connector hub.

The collected swabs were inoculated onto Columbia blood agar plates (Oxoid Ltd) and were incubated aerobically at 35° C. for 48 hours. The swabs were then analysed for bacterial growth. Laboratory staff were blinded as to the source of the sample. The number of colony forming units (CFUs) were reported and the bacteria grown were identified.

64 needle-free connectors and 19 three-way taps were swabbed. Six of the needle-free connectors (9.5%) showed bacterial growth. Coagulase-negative Staphylococci were identified in all six cases. None of the three-way taps showed bacterial growth.

These results show that exterior disinfection of connectors such as needle-free connectors can be insufficient to prevent bacterial colonisation.

### Example 2

### Laboratory Study

A blinded controlled trial was undertaken to evaluate the potential for bacterial transmission through needle-free connectors and three-way taps. 20 needle-free connectors (SmartSite®, Carefusion) and 20 three-way taps (Becton Dickinson) were contaminated with common critical care pathogens by inserting a syringe tip contaminated with critical care pathogen broth into the connector hub. The syringe was removed and the connector hub was left at room temperature for 10 minutes to allow bacterial growth. The composition of the pathogen broth is shown in Table 1.

TABLE 1
Pathogens in the critical care pathogen
broth and their concentrations

| Organism | Concentration |
|---|---|
| E. Coli | 10⁷ CFU/ml |
| Klebsiella oxytoca | 10⁷ CFU/ml |
| Serratia marcescens | 10⁷ CFU/ml |
| Enterobacter cloacae | 10⁷ CFU/ml |
| Pseudomonas aeruginosa | 10⁷ CFU/ml |
| Group G Strep | 10⁷ CFU/ml |
| Group C Strep | 10⁷ CFU/ml |
| MSSA | 10⁷ CFU/ml |
| MRSA | 10⁷ CFU/ml |
| Entercoccus faecalis | 10⁷ CFU/ml |
| Entercoccus faecium (GRE) | 10⁷ CFU/ml |

All connectors were then cleaned with 2% chlorhexidine in 70% isopropyl wipes (Sani-Cloth®, PDI®) in accordance with the manufacturer's instructions. 5 ml sterile saline was injected through each connector and collected at the “patient” end of the apparatus into growth media. These samples were incubated aerobically at 35° C. for 48 hours and then plated onto blood agar.

Plates were analysed by a blinded microbiologist. Growth was calculated as number of colony forming units (CFU). Heavy growth was judged as >100 CFU; light growth as 5-100 CFU; and no growth as <5 CFU. Fisher's exact test was used for comparisons.

The needle-free connectors showed heavy contamination in 19 out of 20 samples, whereas the three-way taps showed heavy contamination in 6 out of 20 samples (P<0.001). No samples showed light contamination.

These results show decontamination of contaminated connector hubs can be difficult to achieve using standard disinfection procedures, in particular for needle-free connectors.

### Example 3

### Preparation of Coated Syringe

Using a sterile non-touch technique, standard 5 ml luer slip syringes (Becton Dickinson) were filled with 5 ml sterile normal (0.9%) saline. To prepare the coated syringe, the external luer of the syringe tip was coated with 4% liquid chlorhexidine, but not the front of the tip. The coating was then dried by placing the syringe in an incubator at 35° C. for 30 minutes. The application was repeated once.

### Example 4

### Decontamination of Hub with Coated Device

20 needle-free connectors and 20 three-way taps were contaminated with common critical care pathogens as described in Example 2. The hubs were then swabbed to demonstrate equal bacterial contamination in both groups.

A pre-filled syringe prepared according to Example 3 was inserted into the injector hubs at time 0, 1 hour, 2 hours, 3 hours and 4 hours, and sterile normal (0.9%) saline was injected through the connector to “the patient”. This was also carried out with control (non-coated) syringes. The internal surface of the connector hubs were swabbed at time 0, 1 hour and 4 hours to determine the concentration of bacteria remaining in the hub. Swabs were inoculated onto blood agar plates and incubated aerobically for 48 hours at 35° C.

Plates were analysed by a blinded microbiology technician. Growth was calculated as number of colony forming units (CFU). Heavy growth was judged as >100 CFU; light growth as 5-100 CFU; and no growth as <5 CFU. A one-tailed Fisher's exact test was used for comparisons.

The results are shown in FIGS. 6 and 7. FIG. 6 shows the reduction in bacterial growth seen in needle-less connectors comparing the normal syringe and the coated syringe. FIG. 7 shows the reduction in bacterial growth seen in three-way connectors comparing the standard syringe and the coated syringe.

Baseline swabbing demonstrated 100% bacterial hub colonisation in both connectors, (CFU>100). The coated syringe showed a significant reduction in CFU growth at 0 and 1 hours compared with control syringes (P<0.05). At 4 hours, the coated syringe completely eliminated bacterial growth in both of the connector hubs. These results show that the coated syringe can effectively disinfect both three-way and needle-less connectors.

### REFERENCES

Ramritu P, Halton K, Cook D, Whitby M, Graves N. Catheter-related bloodstream infections in intensive care units: a systematic review with meta-analysis. J Adv Nurs. 2008; 62(1): 3-21.
Richardson J, Hodges E, Rogerson G, Mariyaselvam M, Maduakor C, Young P. Bacterial colonisation and transmission risk of intravenous connectors. J Infect Prev. 2013; 14: S3 Abstract ID 2575.
Maragakis L L, Bradley K L, Song X, et al. Increased catheter related bloodstream infection rates after the introduction of a new mechanical valve intravenous access port. Infect Control Hosp Epidemiol 2006; 27:67-70.
Jarvis W R, Murphy C, Hall K K, et al. Health Care—Associated Bloodstream Infections Associated with Negative- or Positive-Pressures or Displacement Mechanical Valve Needleless Connectors. Clin Infect Dis 2009; 49:1821-7.
Yebenes J C, Vidaur L, Serra-Prat M, et al. Incidence of catheter-related bloodstream infection in critically ill patients using a disinfectable, needle-free connector: a randomized controlled trial. Am J Infect Control 2004; 32:291-295.
Esteve F, Pujol M, Limon E et al. Bloodstream infection related to catheter connections: a prospective trial of two connections systems. J Hosp Infect 2007; 67:30-34.
Yebenes J C, Delagado M, Goretti S et al. Efficacy of three different valve systems of needle-free closed connectors in avoiding access of microorganisms to endovascular catheters after incorrect handling. Crit Care Med 2008; 36(9):2558-2561.
Austin P, Elia M. Improved aseptic technique can reduce variable contamination rates of ward-prepared parenteral doses. J Hosp Infect. 2013; 83(2):160-163.
Kerenyi, Borza Z, Csontos C et al. Impact of medications on bacterial growth in syringes. J Hosp Infect. 2011; 79:265-266.
van Grafhorst J P, Foudraine N A, Nooteboom F, Crombach W H, Oldenhof N J, van Doorne H. Unexpected high risk of contamination with staphylococci species attributable to standard preparation of syringes for continuous intravenous drug administration in a simulation model in intensive care units. Crit Care Med 2002; 30(4):833-6.
Stucki C, Sautter A, Favet J and Bonnabry P. Microbial contamination of syringes during preparation: The direct influence of environmental cleanliness and risk manipulations on end-product quality. Am J Health Syst Pharm 2009; 66(22): 2032-6.
Magee L, Godsiff L, Matthews I, Farrington M, Park G R. Anaesthetic drugs and bacterial contamination. Eur J Anaesthesiol Suppl. 1995; 12:41-3.

1. A syringe for disinfecting a port of a medical device in fluid communication with a patient, the syringe comprising a barrel and a tip, the syringe tip being adapted for connection to the port, wherein:
the external surface of the syringe tip is coated with a composition in a solid or solid-like phase that comprises a disinfectant, and wherein the disinfectant composition is able to release the disinfectant upon contact with a liquid.

2. The syringe according to claim 1, wherein the port comprises a connector hub.

3. The syringe according to claim 1 or 2, wherein the medical device comprises a catheter and/or a cannula.

4. The syringe according to claim 1 or 2, wherein the medical device comprises a connecting tube.

5. The syringe according to claim 1, wherein the external surface of the syringe tip is coated with a composition in a solid phase.

6. The syringe according to claim 1, wherein the disinfectant composition exclusively coats the external surface of the syringe tip.

7. The syringe according to claim 1, wherein the entire surface area of the external surface of the syringe tip is coated with the disinfectant composition.

8. The syringe according to claim 1, wherein a fraction of the surface area of the external surface of the syringe tip is coated with the disinfectant composition.

9. The syringe according to claim 8, wherein one half of the external surface of the syringe tip is coated with the disinfectant composition.

10. The syringe according to claim 1, wherein the barrel of the syringe is coated with the disinfectant composition.

11. The syringe according to claim 1, wherein the syringe tip is adapted for the formation of a luer connection with the port.

12. The syringe according to claim 11, wherein the luer connection is a luer lock connection.

13. The syringe according to claim 11, wherein the luer connection is a luer slip connection.

14. The syringe according to claim 11, wherein the syringe tip is adapted for the formation of a male luer connection and the port is adapted for the formation of a female luer connection.

15. The syringe according to claim 1, wherein the disinfectant composition is able to release the disinfectant upon contact with a liquid by the disinfectant dissolving or dispersing in the liquid.

16. The syringe according to claim 1, wherein the disinfectant comprises one or more of a biocidal agent, a sporicidal agent, a bactericidal agent or a viricidal agent.

17. A process for disinfecting a port of a medical device in fluid communication with a patient comprising:
(a) placing the syringe according to claim 1 into the port; and
(b) contacting the disinfectant composition with a liquid, thereby releasing the disinfectant.

18. The process according to claim 17, wherein steps (a) and (b) take place simultaneously.

19. A kit comprising the syringe according to claim 1 and a connector hub for use with a medical device to be placed in fluid communication with a patient.

20. (canceled)
